(19) United States
(12) Patent Application Publication
(10) Pub. No.: US 2004/0087936 A1
(43) Pub. Date: May 6, 2004

(54) System and method for treating abnormal tissue in an organ having a layered tissue structure

(75) Inventors: Roger A. Stern, Cupertino, CA (US); Robert A. Ganz, Minneapolis, MN (US); Brian D. Zelickson, Minneapolis, MN (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: BARRx, Inc., Sunnyvale, CA (US)

(21) Appl. No.: 10/426,923

(22) Filed: Apr. 29, 2003

Related U.S. Application Data
(63) Continuation-in-part of application No. 10/370,645, filed on Feb. 19, 2003.
Application No. 10/370,645 is a division of application No. 09/714,344, filed on Nov. 16, 2000, now Pat. No. 6,551,310.
(60) Provisional application No. 60/377,336, filed on Apr. 30, 2002.

(51) Int. Cl. A61B 18/14

(52) U.S. Cl. 606/41

(57) ABSTRACT

A method, and system, are provided treating a tissue site of a tissue structure that has at least a first and a second tissue plane. An energy delivery device is provided. A barrier is created between the first and second tissue planes. At least a portion of an energy delivery device is positioned at the tissue site. Sufficient energy is delivered from the energy delivery device to create cell necrosis of at least a portion of the first tissue plane.

### CROSS-REFERENCE TO RELATED APPLICATIONS

The application is a continuation in part of commonly assigned, co-pending U.S. patent application Ser. No. 10/370,645 filed Feb. 19, 2003, which is a divisional of Ser. No. 09/714,344 filed Nov. 16, 2000. This application also claims the benefit of priority from commonly assigned, co-pending U.S. Provisional Patent Application Ser. No. 60/377,336 filed Apr. 30, 2002. All applications listed above are fully incorporated herein by reference for all purposes.

### FIELD OF THE INVENTION

This invention relates generally systems and methods for treating abnormal tissue in an organ that has at least two tissue planes, and more particularly to systems and methods for treating the epithelium in an esophagus including the use of a barrier layer between a tissue to be treated and deeper tissues.

### BACKGROUND OF THE INVENTION

Those with persistent problems or inappropriate relaxation of the lower esophageal. Sphincter can develop a condition known as gastroesophageal reflux disease, manifested by classic symptoms of heartburn and regurgitation of gastric and intestinal content. The causative agent for such problems may vary. Patients with severe forms of gastroesophageal reflux disease, no matter what the cause, can sometimes develop secondary damage of the esophagus due to the interaction of gastric or intestinal contents with esophageal cells not designed to experience such interaction.

The treatment of gastroesophageal reflux disease, caused by malfunction of the lower esophageal sphincter, is not the subject of this patent application, rather the invention is focused on treatment of the secondary damage to esophageal tissue particularly a condition known as Barrett's esophagus.

The esophagus is composed of three tissue layers; a superficial mucosal layer lined by squamous epithelial cells, a middle submucosal layer and a deeper muscle layer. When gastroesophageal reflux occurs, the superficial squamous epithelial cells are exposed to gastric acid, along with intestinal bile acids and enzymes. This exposure may be tolerated, but in some cases can lead to damage and alteration of the squamous cells, causing them to change into taller, specialized columnar epithelial cells. This metaplastic change of the mucosal epithelium from squamous cells to columnar cells is called Barrett's esophagus, named after the British surgeon who originally described the condition.

Barrett's esophagus has important clinical consequences, since the Barrett's columnar cells can, in some patients, become dyplastic and then progress to a certain type of deadly cancer of the esophagus. The presence of Barrett's esophagus is the main risk factor for the development of adenocarcinoma of the esophagus.

Accordingly, attention has been focused on identifying and removing this abnormal Barrett's columnar epithelium in order to mitigate more severe implications for the patient. Examples of efforts to properly identify Barrett's, epithelium or more generally Barrett's esophagus, have included conventional visualization techniques known to practitioners in the field. Although certain techniques have been developed to characterize and distinguish such epithelium cells, such as disclosed in U.S. Pat. Nos. 5,524,622 and 5,888,743, there has yet to be shown safe and efficacious means of accurately removing undesired growths of this nature from portions of the esophagus to mitigate risk of malignant transformation.

Devices and methods for treating abnormal body tissue by application of various forms of energy to such tissue have been described, and include laser treatment, microwave treatment, radio-frequency ablation, ultrasonic ablation, photodynamic therapy using photo-sensitizing drugs, argon plasma coagulation, cryotherapy, and x-ray. These methods and devices are all defective however, since they do not allow for precise control of the depth of penetration of the energy means. This is a problem since uncontrolled energy application can penetrate too deeply into the esophageal wall, beyond the mucosa and submucosal layers, into the muscularis externa, potentially causing esophageal perforation, stricture or bleeding. In addition, most of these methods and devices treat only a small portion of the abnormal epithelium, making treatment of Barrett's time-consuming, tedious, and costly.

For example, International Patent Application Number PCT/US/00/08612 describes a method and apparatus for treating body structures, involving unwanted features or other disorders. In one embodiment of that invention, a treatment device and method is described for treating a portion of the mucosal surface of the esophagus using the application of energy or other means. The device and method for treating the esophagus describes treating a limited arc of the esophageal tissue at a time and does not provide application of energy to effect ablation of tissue to a controlled depth.

In many therapeutic procedures in general, it is desirable to create a treatment effect in superficial layers of tissue, while preserving intact the function of deeper layers. In many tissues, for example, the esophagus, natural layers are present. As mentioned above, in the esophagus, there are three layers, the mucosal layer, the submucosa, and the deeper muscularis layer. In other tubular organs, many times a fourth, outer layer called the serosa, is also present. In the treatment of various disease conditions, for example, Barrett's esophagus, it is desired to treat one or more of the more superficial layers while preserving the function of the deeper layers. This is to ensure complete treatment of the desired tissue layers, the target tissue, while preserving, the function of the deeper structures. In the treatment of Barrett's esophagus, the consequences of treating too deeply and affecting layers beneath the mucosa can be significant. For example, treating too deeply and affecting the muscularis can lead to perforation or the formation of strictures. In the treatment of Barrett's esophagus, it may be desired to, treat the innermost mucosal layer, while leaving the intermediate submucosa intact. In some situations, it may be desired to treat both the mucosal and submucosa layers, while leaving the muscularis layer intact.

The superficial layer (mucosa) may be treated, for example, to destroy the tissue of the layer, using therapeutic delivery of destructive energies to the tissue. In order to prevent the unwanted destruction of the deeper tissue layers the level of the destructive energy applied to the target tissue is often reduced to more adequately ensure that the deeper layers are not treated. This reduction in treatment energy may prevent adequate destruction of the target tissue. This reduction in therapeutic energy delivery is not without a price. In particular, the reduction in therapeutic energy delivery may allow some of the targeted tissue layer to survive destruction, possibly resulting in the need for additional treatments.

For example, where radio frequency energy is used to ablate the superficial mucosal layer, an energy delivery level sufficient to ensure the ablation of the mucosal layer might also harm the deeper submucosal layer and/or muscle layer. Reducing the energy delivery level so as to leave the submucosal layer and muscle layer substantially unharmed may allow some of the mucosal tissue layer to survive the ablation. Controlling the delivery of the energy to the target tissue layer may be quite difficult given the variable nature of the esophagus itself and the variable nature of the individual tissue layers, both between patients and over the length of a single esophagus.

What would be desirable is a method and device for ensuring complete ablation of an inner layer while ensuring that the deeper layers are unharmed.

### SUMMARY OF THE INVENTION

Accordingly, an object of the present invention is to provide systems and methods for treating abnormal tissue in an organ that has at least two tissue planes.

Another object of the present invention is to provide systems and methods for treating the epithelium in an esophagus.

Yet another object of the present invention is to provide systems and methods for treating mucosal tissue of the esophagus while preserving muscularis tissue.

Another object of the present invention is to provide systems and methods for treating mucosal and submucosal tissue of the esophagus while preserving muscularis tissue.

A further object of the present invention is to provide systems and methods for treating esophagus tissue including the use of a barrier layer between a tissue to be treated and deeper tissues.

Another object of the present invention is to provide systems and methods for treating esophagus tissue utilizing RF energy and the use of a barrier layer.

These and other objects of the present invention are achieved in a method of treating a tissue site of a tissue structure that has at least a first and a second tissue plane. An energy delivery device is provided. A barrier is created between the first and second tissue planes. At least a portion of an energy delivery device is positioned at the tissue site. Sufficient energy is delivered from the energy delivery device to create cell necrosis of at least a portion of the first tissue plane.

In another embodiment of the present invention, a method of treating an esophagus tissue site with at least a first and a second tissue plane is provided. An energy delivery device is introduced through an oral cavity and into the esophagus. A barrier is created between the first and second tissue planes. At least a portion of an energy delivery device is positioned at the esophagus tissue site. Energy is delivered from the energy delivery device at different times to create cell necrosis of at least a portion of the first tissue plane.

In another embodiment of the present invention, a method of treating an esophagus tissue site with at least a first and a second tissue plane. An energy delivery device is introduced through an oral cavity and into the esophagus. A barrier is created between the first and second tissue planes. At least a portion of an energy delivery device is positioned at the esophagus tissue site. Energy is delivered from the energy delivery device to create a controlled cell necrosis of at least a portion of the first tissue plane while minimizing permanent damage to esophageal muscularis tissue.

In another embodiment of the present invention, a method of treating an esophagus tissue site with at least a first and a second tissue plane is provided. An energy delivery apparatus is introduced through an oral cavity and into the esophagus. The energy delivery apparatus includes a plurality of RF electrodes. A width of each RF electrode and a spacing between adjacent RF electrodes are selected to provide a selectable ablation of an esophagus mucosal tissue. A barrier is created between the first and second tissue planes. At least a portion of an energy delivery device is positioned at the esophagus tissue site. Energy is delivered from the energy delivery device to create a controlled cell necrosis of at least a portion of the first tissue plane while minimizing permanent damage to a muscularis tissue.

In another embodiment of the present invention, an apparatus is provided for separating tissue planes at a tissue site. An instrument body has an elongated shaft portion sized and constructed for insertion through an endoscope and into the interior of the tissue site of a patient. A separation mechanism at a distal end of the elongated shaft is attachable to at least a portion of the tissue site. The separation mechanism provides a separation of at least one tissue plane from a second tissue plane. An injection device is coupled to the instrument body.

In another embodiment of the present invention, a system for creating cell necrosis from a tissue site at a human esophagus is provided. A cell necrosis device is provided. An injection catheter injects a barrier material to separate esophageal tissue layers by flowing between tissue layers or expanding one of the tissue layers. The injection catheter has a lumen therethrough, a proximal region, a proximal port, a distal region, and an injection orifice at a distal region and in fluid communication with the lumen. A fluid supply is coupled to the injection catheter proximal port for forcing the barrier fluid through the injection catheter distal orifice.

### BRIEF DESCRIPTION OF THE DRAWINGS

FIG. 1 is a schematic view of portions of an upper digestive tract in a human.

FIG. 2 is a schematic view of a device of the invention, in an expanded mode, within an esophagus.

FIG. 3 is a schematic view of a device of the invention.

FIG. 4 is another view of the device of FIG. 3.

FIG. 5 is a view of a device of the invention.

FIG. 6 shows the electrode patterns of the device of FIG. 3.

FIG. 7 shows electrode patterns that may be used with a device of the invention.

FIG. 8 is a schematic view of another embodiment of a device of the invention.

FIG. 9 shows a top view and a bottom view of an electrode pattern of the device of FIG. 8.

FIG. 10A is a perspective view of the distal region of a first barrier material injection device having closable opposed jaws for grasping and retracting a tissue layer to create a potential space between the retracted layer and a deeper layer, and a longitudinally slideable needle for injecting barrier material into the potential space;

FIG. 10B is a side view of the device of FIG. 10A, having the jaws in a fully closed position;

FIG. 10C is a side view of the device of FIG. 10A, having the jaws in an open position, with the injection needle retracted, approaching the tissue layer to be grasped;

FIG. 10D is a side view of the device of FIG. 10A, having the jaws closed about and retracting a tissue portion to create a potential space, and having the injection needle inserted into the potential space;

FIG. 11A is a side view of one barrier injection device proximal region suitable for coupling to device 200 of FIGS. 10A-10D, shown in a first position, having jaws open and injection needle retracted;

FIG. 11B is a side view of the device proximal region of FIG. 11A shown in a second position, with the jaws closed and the injection needle still retracted;

FIG. 11C is a side view of the device proximal region of FIG. 11A shown in a third position having the jaws closed and the needle distally extending;

FIG. 12A is a perspective view of another barrier material injection device having a distal plate or ring coupled to an outer tube, having an inner sheath for slideably disposing a retraction catheter and/or needle and an injection catheter and/or needle within;

FIG. 12B is a fragmentary side view of the device of FIG. 12A, having the plate disposed against the superficial tissue layer;

FIG. 12C is a fragmentary side view of the device of FIG. 12B, having the retraction needle penetrating the superficial tissue layer;

FIG. 12D is a fragmentary side view of the device of FIG. 12B, having the retraction needle retracting the superficial tissue layer to create a potential space between the superficial and deeper tissue layers, and having the injection needle inserted into the potential space;

FIG. 12E is a fragmentary side view of the retraction needle and injection needle of FIG. 12A, with the retraction needle shown in the distally extended position; and

FIG. 12F is a fragmentary side view of the retraction needle and injection needle of FIG. 12A, with the retraction needle shown in the distally extended position.

FIG. 13 is a photograph of a histologic specimen (pig) demonstrating the separation of esophageal layers (mucosa/submucosa from muscle) using a barrier fluid.

### DETAILED DESCRIPTION OF THE INVENTION

In various embodiments, the present invention provides methods and systems for treating a tissue site of a tissue structure that has at least a first and a second tissue plane. It will be appreciated that the present invention is applicable to a variety of different tissue sites and organs, including but not limited to the esophagus. A treatment apparatus including an energy delivery device is provided. A barrier is created between the first and second tissue planes. At least a portion of an energy delivery device is positioned at the tissue site. Sufficient energy is delivered from the energy delivery device to create cell necrosis of at least a portion of the first tissue plane.

In various embodiments, the present invention provides methods and devices for treating a variety of different types of tissues, including those of the esophagus, as set forth in U.S. Provisional Application No. 60/165,687, filed Nov. 16, 1999, and in International Application No. PCT/USOO/31561, filed Nov. 16, 2000 and published as Publication No. WO 01/35846 A1 on May 25, 2001, and in another International Application No. PCT/US01/15680, filed on May 16, 2001. The disclosures of each of the above is hereby incorporated by reference in their entirety.

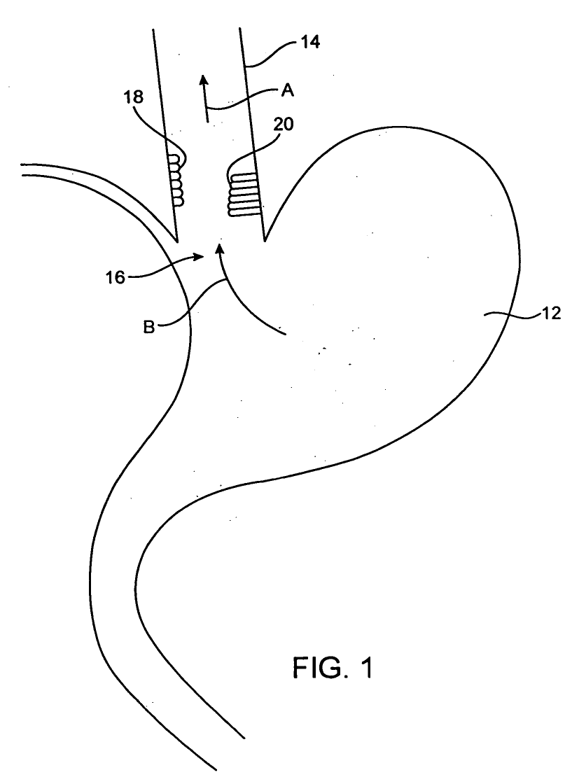

Certain disorders can cause the retrograde flow of gastric or intestinal contents from the stomach 12 into the esophagus 14, as shown in FIG. 1 and represented by arrows A and B. Although the causation of these problems are varied, this retrograde flow may result in secondary disorders, such as Barrett's esophagus, which require treatment independent of and quite different from treatments appropriate for the primary disorder—such as disorders of the lower esophageal sphincter 16. Barrett's esophagus is an inflammatory disorder in which the stomach acids, bile acids and enzymes regurgitated from the stomach and duodenum enter into the lower esophagus causing damage to the esophageal mucosa. Indeed, when this type of retrograde flow occurs frequently enough, damage may occur to esophageal epithelial cells 18. In some cases the damage may lead to the alteration of the squamous cells, causing them to change into taller specialized columnar epithelial cells. This metaplastic change of the mucosal epithelium from squamous cells to columnar cells is called Barrett's esophagus. Although some of the columnar cells may be benign, others may result in adenocarcinoma.

In one embodiment, the present invention provides methods and systems for identifying and removing columnar epithelium of selected sites of the esophagus in order to mitigate more severe implications for the patient. Examples of efforts to properly identify these growths, referred to as Barrett's epithelium or more generally as Barrett's esophagus, have included conventional visualization techniques known to practitioners in the field. Although certain techniques have been developed to characterize and distinguish such epithelium cells there has yet to be shown safe and efficacious means of accurately removing undesired growths and abnormal tissue of this nature from portions of the esophagus to mitigate risk of malignant transformation.

With the present invention, a variety of different energy delivery devices can be utilized to achieve the ablation and cell necrosis, as described hereafter.

In one specific embodiment, the cell necrosis can be achieved with the use of energy, such as radiofrequency energy, at appropriate levels to accomplish ablation of mucosal or submucosal level tissue, while substantially preserving muscularis tissue. Such ablation is designed to remove the columnar growths 20 from the portions of the esophagus 14 so affected. The term “ablation” as used herein means thermal damage to the tissue causing tissue necrosis.

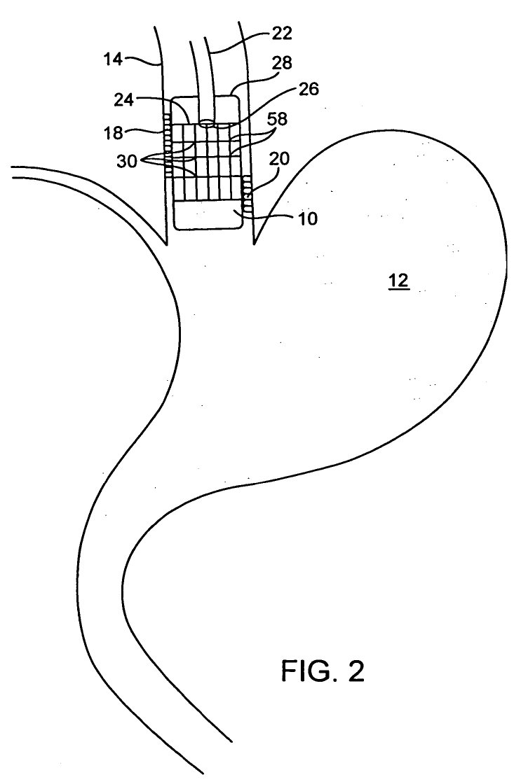

In one embodiment, illustrated in FIG. 2, a treatment apparatus 10 includes an elongated shaft 22, which can be flexible, that is configured to be inserted into the body in any of various ways selected by the medical provider. Shaft 22 may be placed, (i) endoscopically, e.g. through esophagus 14, (ii) surgically or (iii) by other means.

When an endoscope (not shown) is used shaft 22 can be inserted in the lumen of the endoscope, or shaft 22 can be positioned on the outside of the endoscope. Alternately, an endoscope may be used to visualize the pathway that shaft 22 should follow during placement, as well as after removal of the endoscope shaft 22 can be inserted into esophagus 14.

An energy delivery device 24 is provided and can be positioned at a distal end 26 of shaft 22 to provide appropriate energy for ablation as desired. In various embodiments, energy delivery device 24 is coupled to an energy source configured for powering energy delivery device 24 at levels appropriate to provide the selectable ablation of tissue to a predetermined depth of ablation.

Energy delivery device 24 can deliver a variety of different types of energy including but not limited to, radio frequency, microwave, ultrasonic, resistive heating, chemical, a heatable fluid, optical including without limitation, ultraviolet, visible, infrared, collimated or non-collimated, coherent or incoherent, or other light energy, and the like. It will be appreciated that the energy, including but not limited to optical, can be used in combination with one or more sensitizing agents.

In one embodiment, shaft 22 includes a cable that contains a plurality of electrical conductors surrounded by an electrical insulation layer, with an energy delivery device 24 positioned at distal end 26. A positioning and distending device can be coupled to shaft 22. The positioning and distending device can be configured and sized to contact and expand the walls of the body cavity in which it is placed, by way of example and without limitation, the esophagus. The positioning and distending device can be at different positions of energy delivery device 24, including but not limited to its proximal and/or distal ends, and also at its sides.

Energy delivery device 24 can be supported at a controlled distance from, or in direct contact with the wall of the tissue site. This can be achieved by coupling energy delivery device 24 to an expandable member 28. Suitable expandable members 28 include but are not limited to a balloon, compliant balloon, balloon with a tapered geometry, basket, plurality of struts, an expandable member with a furled and an unfurled state, one or more springs, foam, bladder, backing material that expands to an expanded configuration when unrestrained, and the like.

Expandable member 28 can be utilized to regulate and control the amount of energy transferred to the tissue at the tissue site. This can occur with the esophageal wall. Expandable member 28 can be bonded to a portion of shaft 22 at a point spaced from distal end 26.

In another embodiment, expandable member 28 is utilized to deliver the ablation energy itself. An important feature of this embodiment includes the means by which the energy is transferred from distal end 26 to expandable member 28. By way of illustration, one type of energy distribution that can be utilized is disclosed in U.S. Pat. No. 5,713,942, incorporated herein by reference, in which an expandable balloon is connected to a power source, which provides radio frequency power having the desired characteristics to selectively heat the target tissue to a desired temperature.

Expandable member can be made of a variety of different materials, including but not limited to an electroconductive elastomer such as a mixture of polymer, elastomer, and electroconductive particles, a nonextensible bladder having a shape and a size in its fully expanded form, which will extend in an appropriate way to the tissue to be contacted.

In another embodiment, an electroconductive member can be formed from an electrically insulating elastomer, with an electroconductive material, such as copper, deposited onto a surface. An electrode pattern can then be etched into the material, and then the electroconductive member can be attached to an outer surface of a balloon.

In one embodiment, the electroconductive member, which can be a balloon 28, has a configuration that is expandable in the shape to conform to the dimensions of the expanded (not collapsed) inner lumen of the tissue site or structure, such as the human lower esophageal tract. In addition, this electroconductive member can include a plurality of electrode area segments 30. One or more sensors, including but not limited to thermal and the like, can be included and associated with each segment in order to monitor the temperature from each segment and then controlled. The control can be by way of an open or closed feedback system. In another embodiment, the electroconductive member can be configured to permit transmission of microwave energy to the tissue site. In yet another embodiment, a balloon 26 can carry a heat transfer medium, such as a heatable fluid, in one or more portions of balloon 26. In this manner, the thermal energy of the heatable fluid can be used as the ablation energy source. Treatment apparatus can also include steerable and directional control devices, a probe sensor for accurately sensing depth of ablation, and the like.

Energy delivery device 24 can be at a location within the volume of balloon 26. Balloon 26 can also be utilized to place energy delivery device 24, as well as to anchor the position of energy delivery device 24. This can be achieved with balloon 26 itself, or other devices that are coupled to balloon 26 including but not limited to an additional balloon, a plurality of struts, a bladder, and the like.

As shown in FIGS. 4, 5, and 8, in an embodiment of the present invention, energy delivery device 24 can be positioned so that energy is uniformly applied to all or a portion of the circumference of the inner lumen of the esophagus where ablation is desired. This can be accomplished by positioning energy delivery device 24 on the outside circumference of expandable member 26. This same result can be achieved with any of the energy delivery devices 24 utilized, and their respective forms of energy, with respect to expandable member 28 so that the energy is uniformly applied to all or a portion of the circumference of the inner lumen of the esophagus or other tissue site. One way to ensure that the energy is uniformly applied to the circumference of the inner lumen of the esophagus is the use of a vacuum or suction element to “pull” the esophageal wall, or other tissue site, against the outside circumference of expandable member 28. This suction element may be used alone to “pull” the esophageal wall into contact with energy delivery device 24, carried on or by shaft 22 without the use of expandable member 22, or in conjunction with expandable member 28 to ensure that the wall is in contact energy delivery device 24 carried on the outside of expandable member 28.

As described below, the energy source may be manually controlled by the user and is adapted to allow the user to select the appropriate treatment time and power setting to obtain a controlled depth of ablation. The energy source can be coupled to a controller (not shown), which may be a digital or analog controller for use with the energy source, including but not limited to an RF source, or a computer with software. When the computer controller is used it can include a CPU coupled through a system bus. The system may include a keyboard, a disk drive, or other non-volatile memory system, a display and other peripherals known in the art. A program memory and a data memory will also be coupled to the bus.

The depth of ablation obtained with apparatus 10 can be controlled by the selection of appropriate treatment parameters by the user as described in the examples set forth herein. A probe sensor may also be used with the system of the present invention to monitor and determine the depth of ablation.

In one embodiment, apparatus 10 is utilized in a method to treat Barrett's esophagus. This method can include the detection and diagnosis of undesired columnar epithelium within the esophagus. After determining that the portion or portions of the esophagus having this undesired tissue should be partially ablated, then the patient is prepared as appropriate according to the embodiment of the device to be utilized. The practitioner prepares the patient as appropriate and inserts, in one embodiment, via endoscopic access and control, apparatus 10 through the mouth of the patient. As discussed above, apparatus 10 can be inserted through a channel of the endoscope, located on the outside of and along the side of the endoscope. Apparatus 10 can also be inserted through the mouth of the patient to the desired location in the esophagus without an endoscope after an endoscope has been used to identify the proper location and identify the path for insertion of the device.

In one embodiment, apparatus 10 is inserted with the endoscope. After apparatus 10 is inserted, further positioning of portions of apparatus can occur until proper location and visualization identifies the ablation site in the esophagus. Selection and activation of energy delivery device 24, which can be an entire or partial circumferential electrode array, or the appropriate quadrant(s) or portion(s)/segment(s) of the array is performed by the physician, including appropriate power and time settings according to the depth of ablation desired. Additional settings may be necessary as further ablation is required at different locations and/or at different depths within the patient's esophagus. Following the ablation, appropriate follow-up procedures as are known in the field are accomplished with the patient during and after removal of the device from the esophagus.

In one method of the invention, following the ablation treatment to remove the Barrett's epithelium, the patient is treated with acid suppression therapy, which has been shown to enhance the growth of normal epithelium during the healing process.

The ablation treatment with optical energy may also be accompanied by improved sensitizer agents, such as hematoporphyrin derivatives such as Photoffine® (porfimer sodium, registered trademark of Johnson & Johnson Corporation, New Brunswick, N.J.).

In yet another embodiment of a method of the present invention, apparatus 10 may be utilized as a procedural method of treating dysplasia or cancerous tissue in the esophagus. After determining that the portion or portions of the esophagus with undesired tissue should be partially or fully ablated, then the patient is prepared as appropriate. Treatment is provided as described above.

In yet another method of the present invention, the practitioner may first determine the length of the portion of the esophagus requiring ablation by visual observation through an endoscope. Apparatus 10 can be different sized ablation catheters, each with a different length of energy delivery. By way of illustration, if the practitioner determined that 1 centimeter of length of the esophageal surface required ablation, an ablation catheter having 1 centimeter of length of energy delivery can be selected for the ablation. The length of energy delivery device 24, or other energy distribution means associated with expandable member 28 can vary in length. By way of example, the length can be from approximately 1 to 10 cm.

In yet another embodiment of the present invention, apparatus 10 can be a plurality of ablation catheters, where energy delivery device 24 is associated with a balloon 28 can be provided. The diameter of balloon 28, when expanded, can vary depending on the application, but can be from 12 to 35 mm. The practitioner can select an ablation catheter that has an expanded diameter which can cause the esophagus to stretch, and the mucosal layer to thin out. This reduces blood flow at the site of the ablation. The esophagus normally is 5 to 6 mm thick. With this method of the present invention the esophagus is stretched and thinned so that the blood flow through the esophageal vasculature is occluded. It is believed that by reducing the blood flow in the area of ablation, the heat generated by the radiant energy is less easily dispersed to other areas of the esophagus. This can cause a focusing of the energy to the ablation site.

One method to determine the appropriate diameter of ablation catheter to use with a particular patient would is to first use a highly compliant balloon connected to a pressure sensing device. The balloon is inserted into the esophagus and positioned at the desired site of the ablation, and inflated until an appropriate pressure reading is obtained. The diameter of the inflated balloon is determined and apparatus 10, with the appropriate size of a balloon 28 that is capable of expanding to that diameter is selected. The esophagus can be expanded to a pressure of 60-120 lbs./square inch. In this method of the present invention, it is desirable to expand the expandable electroconductive member 28, such as a balloon, sufficiently to occlude the vasculature of the submucosa, including the arterial, capillary or venular vessels. The pressure to be exerted to do so should be greater than the pressure exerted by such vessels. Alternately, the practitioner may determine the appropriate diameter of the ablation catheter to use with visual observation using an endoscope.

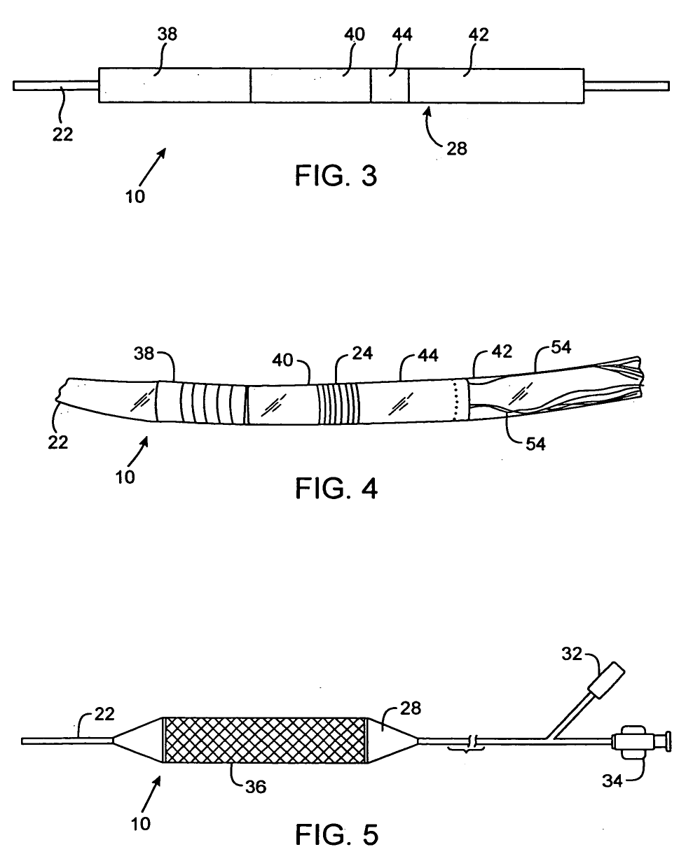

Operation and use of various embodiments of the present invention are described as follows. The embodiments of apparatus 10 are illustrated in FIGS. 3, 4, and 5. As shown in FIG. 5, shaft 22 can be connected to a multi-pin electrical connector 32, which is connected to the power source and can include a male luer connector 34 for attachment to a fluid source useful in expanding expandable member 28.

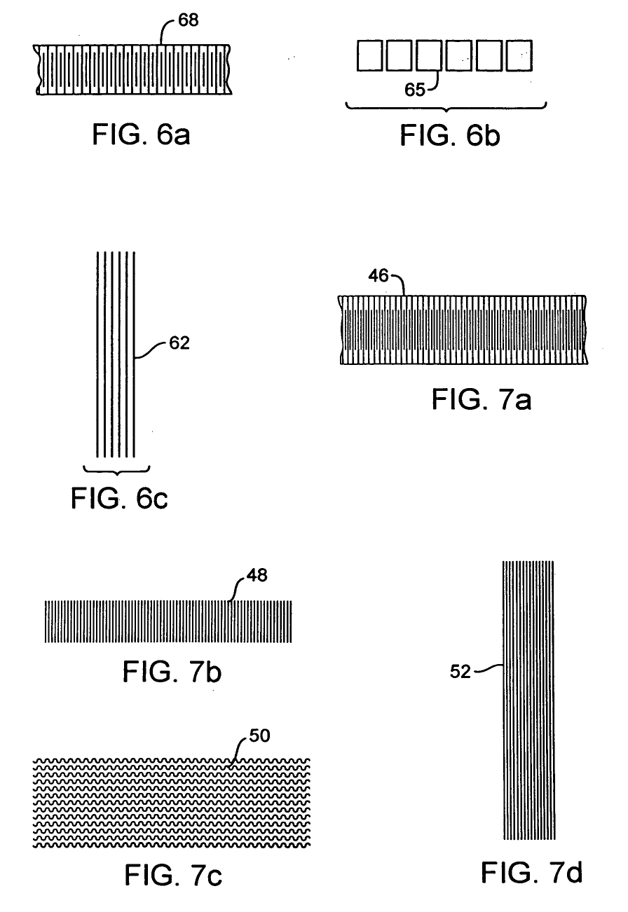

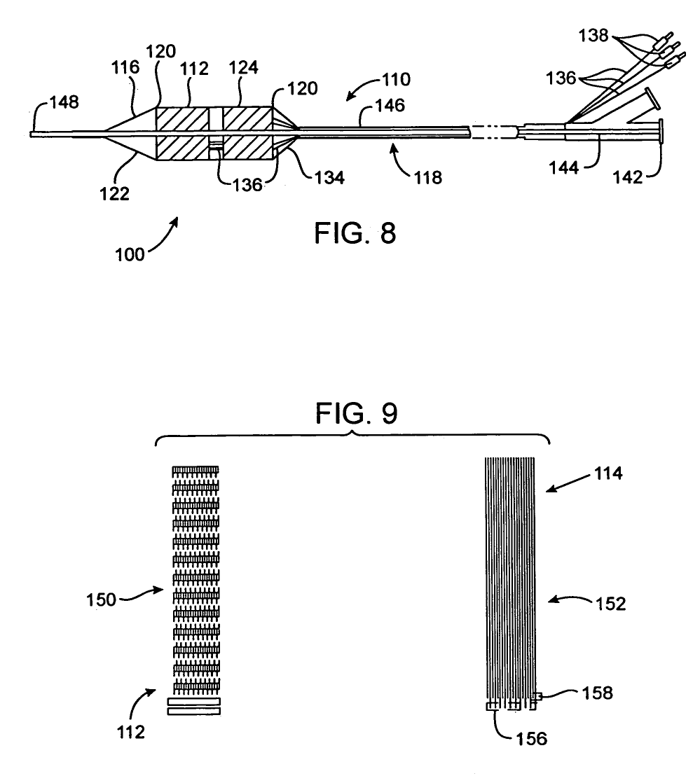

In one embodiment, shaft 22 may have an electrode 36 wrapped around the circumference. In other embodiments, the expandable member of the device shown in FIGS. 3 and 4, which is a balloon 28, further includes three different electrode patterns, the patterns of which are represented in greater detail in FIG. 6. One or more than one electrode pattern can be used in apparatus 10 of the present invention, such as that illustrated in FIG. 8 described below.

In apparatus 10 shown in FIGS. 3 and 4, shaft 22 has six bipolar rings 38, with 2 mm separation at one end of shaft 22, (one electrode pattern). Adjacent to bipolar rings 38 is a section of six monopolar bands or rectangles 40 with 1 mm separation (a second electrode pattern), and another pattern of bipolar axial interlaced finger electrodes 42 is positioned at the other end of shaft 22 (a third electrode pattern). In this device, a null space 44 is positioned between the last of monopolar bands 40 and bipolar axial interlaced finger electrodes 42. Apparatus 10 used in the study was prepared using a polyimide flat sheet of about 1 mil (0.001″) thickness coated with copper. The desired electrode patterns were then etched into the copper. Apparatus 10, in these embodiments, is adapted for use with an RF energy source.

In other embodiments a width of each RF electrode can be no more than, (i) 3 mm, (ii) 2 mm, (iii) 1 mm or (iv) 0.5 mm and the like. A spacing between adjacent RF electrodes can be no more than, (i) 2 mm, (ii) 1 mm, (iii) 0.5 mm and the like. The plurality of electrodes can be arranged in segments, with at least a portion of the segments being multiplexed. An RF electrode between adjacent segments can be shared by each of adjacent segments when multiplexed.

The electrode patterns of the present invention may be varied depending on the length of the site to be ablated, the depth of the mucosa and submucosa, in the case of the esophagus, at the site of ablation and other factors. Other suitable RF electrode patterns which may be used include, without limitation, those patterns shown in FIGS. 7(a) through 7(d) as 46, 48, 50 and 52, respectively. Pattern 46 is a pattern of bipolar axial interlaced finger electrodes with 0.3 mm separation. Pattern 48 includes monopolar bands with 0.3 mm separation. Pattern 52 includes bipolar rings with 0.3 mm separation. Pattern 50 is electrodes in a pattern of undulating electrodes with 0.2548 mm separation.

In certain method and apparatus embodiments where the application of apparatus 10 is to treat the esophagus 14, energy delivery device 24 can include a plurality of electrodes that are positioned at an exterior of a balloon 28 which can have a diameter of about 18 mm. In these embodiments, apparatus 10 is adapted to use RF energy radio frequency by attaching wires 54, see FIG. 4, to electrodes 24 to connect them to the power source.

### EXAMPLE 1

Balloon 28 was deflated and the catheter was inserted into the esophagus 14 as described below. In addition to the series of three different electrode patterns 24, a number of different energy factors can be applied to the esophagus 14 of a normal immature swine (about 25 kgs). First, an endoscope was passed into the stomach of the subject. Apparatus 10 was placed into the distal esophagus using endoscopic guidance. Balloon 28 was inflated to press electrodes 24 against the esophageal mucosa. There was no indication that balloon dilation resulted in untoward effects on the esophagus 14.

Once balloon 28 and electrodes 24 are in place the first set of radio frequency (“RF”) applications are made. Following endoscopic evaluation of the treated areas, apparatus 10 was withdrawn proximally. The placement of balloon 28, and electrodes 24, was endoscopically to assure a gap of normal tissue between the area of the first application and the second application, which gap assures identification of the two treatment areas during post procedure evaluations.

The procedure was repeated a third time using a similar procedure to that of the second application. During, the treatment the tissue impedance was monitored as an indicator of the progress of the treatment, high impedance being an indication of desiccation. Accordingly, the practitioner can determine through monitoring the tissue impedance when sufficient ablation has occurred.

The treatment transformer tap was changed for the bipolar treatments from 50 to 35. Of note was the observation that towards the end of the monopolar treatments, the watts output as reported on the generator was decreased from a setting of 15 watts to a reading of 3 to 4 watts. The increase in impedance observed in the study may be useful as an endpoint for controlling the RF energy at the ablation site.

The RF energy can be applied to the electroconductive members, electrodes 24, in a variety of ways. In one embodiment, it was applied in the bipolar mode to electrodes 24, which were bipolar rings 52 through simultaneous activation of alternating bipolar rings 52. In another embodiment, it was applied to the bipolar rings 52 through sequential activation of pairs of bipolar rings 52. In another embodiment, the RF energy can be applied in monopolar mode through sequential activation of individual monopolar bands or simultaneous activation of the monopolar bands.

After the treatment of the swine esophagus, as described above using radio frequency, the esophagus 14 was extirpated and fixed in 10 percent normal buffered formalin (NBF). Three distinct lesion areas were observed corresponding to the three treatment sites and the esophagus 14 was divided into three sections that approximated the three treatment zones. Each segment was cut into 4 to 5 mm thick serial cross sections. Selected sections from each treatment segment were photographed and the photographs of representative treatment segments were assembled side by side to compare similar catheter electrode patterns among the three treatment regimens.

The following observations were made. Almost all the treated segments demonstrated necrosis of the mucosa. Changes with the submucosal, muscularis and adventitial layers were observed, typically demonstrated by tissue discoloration suggestive of hemorrhage within the tissue. Finally in comparing the tissue to the normal esophageal morphology, most treated segments were dilated with thinned walls. Thus, all electrode 24 patterns and treatment parameters resulted in ablation of the mucosal layer of the esophagus 14.

Another embodiment of an apparatus 100 device of the present invention is shown in FIG. 8. This device comprises an esophageal electrode balloon catheter 110 comprised of two electrode arrays, 112 and 114, respectively, affixed to the outside of an 18.25 mm diameter×40 mm long balloon 116 that is mounted on a 3 foot long catheter 118. One electrode 112 is aligned with an edge 120 that intersects the taper region located at the distal end of balloon 122 while the other 124 is aligned with the proximal intersecting taper edge located at the proximal end of balloon 126.

FIG. 9 shows a bottom view 150 and a top view 152 of electrode arrays 112 and 114. In this embodiment, each array 112 and 114 has 20 parallel bars, 0.25 mm. wide×60-65 mm long, separated by gaps of 0.3 mm. When adhered to balloon 126, the bars on the circuit form twenty complete continuous rings around the circumference. Electrode arrays 112 and 114 can be etched from a laminate consisting of copper on both sides of a polyimide substrate. One end of each copper bar has a small plated through-hole 128, which allows signals to be passed to these bars from 1 of 2 copper junction blocks 130 and 132, respectively, on the back of the laminate. One junction block 130 is connected to all of the even numbered bars, while the other junction block 132 is connected to all of the odd numbered bars.

As shown in FIG. 8, each junction block 130 and 132 is then wired to a bundle of five thirty-four AWG wires 134. The wiring is external to balloon 126, with the distal circuit wires affixed beneath the proximal circuit. Upon meeting the shaft of the catheter, these four bundles 134 can be soldered to three litz wire bundles 136. One bundle 136 serves as a common conductor for both circuits while the other two bundles 136 are wired individually to each of the two circuits. The litz wires are encompassed with heat-shrink tubing along the entire length of the shaft of the catheter. Upon emerging from the proximal end of the catheter, each of these bundles 136 is individually insulated with heat-shrink tubing before terminating to a mini connector plug 138.

The y-connector 142 at the proximal end of the catheter includes access ports for both the thru lumen 144 and the inflation lumen 146. The thru lumen spans the entire length of the balloon catheter and terminates with a flexible lumen tip 148 at the distal end of balloon 126.

For delivery of apparatus 100, balloon 126 is folded and placed within a sheath (not shown). During deployment this sheath is retracted along the shaft to expose balloon 126.

Apparatus 100, illustrated in FIG. 8, is designed for use with the RF energy methods as set forth herein. Electrode arrays 112 and 114 can be activated with approximately 40 watts of radio frequency power for the length of time necessary to deliver from 200 to 600 joules of energy to the tissue. Since the total treatment area of a 1 centimeter long electrode array wrapped around an 18.25 millimeter diameter balloon 126 is about 5.7 square centimeters, this equates to approximately 35 to 105 joules per square centimeter of esophageal area.

For an apparatus 100 employing a different length electrode array 112 or 114, or a different diameter balloon 126, the desired power and energy settings can be scaled as needed to deliver the same power and energy per unit area. These changes can be made either automatically or from user input to the RF power source. If different treatment depths are desired, the geometry of electrode arrays 112 and 114 can be modified to create either a deeper or more superficial treatment region. Making electrode arrays 112 and 114, which can be bipolar electrode rings, more narrow and spacing them closer together reduces the treatment depth. Making electrode arrays 112 and 114 wider, and spacing them further apart, increases the depth of the treatment region. Non-uniform widths and spacings may be exploited to achieve various treatment effects.

As described in one method of the present invention using a device of the present invention, where RF energy is applied to the tissue to be ablated, the depth of ablation may be controlled by proper selection of the treatment settings. For apparatus 100 of FIG. 8, with electrode arrays 112 and 114 having a length of about 1 centimeter long and a diameter of about 18 mm, it is desirable to provide power in the range of 20-60 watts for a time period between 5 and 20 seconds.

In order to ensure good contact between the esophageal wall and electrode arrays 112 and 114, slight suction may be applied to the through-lumen tube to reduce the air pressure in the esophagus 14 distal to balloon 126. The application of this slight suction can be simultaneously applied to the portion of the esophagus 14 proximal to balloon 126. This suction causes the portion of the esophageal wall distended by balloon 126 to be pulled against electrode arrays 112 and 114 located on balloon 126.

Various modifications to the above-mentioned treatment parameters with electrode arrays 112 and 114 can be made to optimize the ablation of the abnormal tissue. To obtain shallower lesions than the ones obtained in the above-mentioned study the RF energy applied may be increased while decreasing the treatment time. To obtain very shallow lesions using apparatus 100 of FIG. 8, with electrode arrays 112 and 114 having a length of about 1 centimeter long and a diameter of about 18 mm, it is desirable to provide power in the range of 300-350 watts for a time period sufficient to deliver between 20-80 Joules of energy. Also, the patterns of electrode arrays 112 and 114 may be modified, such as shown in FIG. 7, to improve the evenness and shallowness of the resulting lesions. The systems and methods of the present invention can also be modified to incorporate temperature feedback, resistance feedback and/or multiplexing electrode channels.

In various embodiments, the methods and apparatus of the present invention can provide a barrier or separation layer between tissue planes of an organ to be ablated, including but not limited to the esophagus 14, where the organ has a lining with multiple tissue layers. Certain embodiments of the present invention include methods for ablating abnormal tissue in a human esophagus 14, where the esophagus 14, as described above, has at least three adjacent tissue layers including a first, most superficial, mucosal layer, a second, submucosal layer disposed beneath the mucosal layer, and a third, muscularis layer disposed beneath the submucosal layer.

In one embodiment of the present invention, a method is provided for separating at least one of the three tissue layers from an adjacent tissue layer to form a separation barrier between the deeper tissue layer and the adjacent more superficial tissue layer. A tissue destructive treatment can then be applied to the more superficial layer, such that the separation barrier attenuates transmission of the treatment to the deeper tissue layer. The tissue destructive treatment can be selected from cryogenic ablation, heat energy, electrical energy, light energy, collimated light energy(laser), non collimated light, ultrasonic energy, microwave energy, and radio frequency energy and photodynamic therapy, as well as using a drug sensitizer in combination with light energy. The sensitizer may be administered topically, orally or intravenously.

In some embodiments of the present invention, the separating step includes separating the mucosal layer from the submucosal layer. In other esophageal applications, the separating step separates the submucosal layer from the muscularis layer. In certain embodiments of the present invention, the separating step includes separating a deeper layer from a more superficial layer by expanding one tissue layer, such as the submucosal layer, into a more superficial portion, adjacent to the mucosa and a deeper portion adjacent the muscle layer. After separating the tissue layers, a tissue destructive treatment energy can be applied in a dosage that would significantly harm the deeper layer but for the presence of the separation barrier. Use of such a separation layer allows a higher energy to be applied to the target tissue, that higher energy may provide a more efficacious treatment than if the separation layer is not present, because the separation layer will protect tissue of the deeper tissue layer from destruction.

In one method of the present invention, the tissue layer separating step includes injecting a fluid, which can be gas, liquid, or combination, in between at least two of the three layers. The fluid is gaseous in some methods and liquid in other methods. Some fluids include a connective tissue weakening agent for improving the separation of the tissue layers. Connective tissue weakening agents useful with certain methods of the present invention include but are not limited to, hyaluronidase, collagenase, elastase, and other known dissociating enzymes.

The barrier fluid can be selected to prevent or attenuate the transmission of the destructive therapeutic energy from the superficial to the deeper layer. The selection of the fluid will thus depend upon the nature of the destructive energy applied. Some fluids are thermally insulating, while other fluids are electrically insulating. Still other fluids are electrically conducting. Ultrasonically reflecting fluids are used in some applications, while other applications use a fluid that is either optically reflecting or absorbing.

In some methods, an injection catheter having a lumen there through is provided. The injection catheter can have a sharp distal tip, which may be a needle mechanism, for injecting the fluid into the esophageal layers. The injection catheter distal tip can be inserted to a depth located between layers to be separated. The fluid can then be injected between the layers, and the layers allowed to separate. The placement of the injection catheter distal tip can be determined by using a barrier fluid that comprises an agent that allows the movement of the fluid within the tissue layers and/or between tissue layers to be followed by the practitioner. For example, the barrier fluid may be colored with methylene blue or food coloring and visually or optically detected.

In some methods, at least one, and preferably at least four radially spaced injections of barrier fluid are made per injection region. An injection region is a circumferential portion of the esophagus where treatment energy will be applied. The radially spaced injections can be longitudinally spaced between about 0.5 and2 cm apart. Each injection site can have between about 1 and10 cubic cm of a fluid injected.

In one embodiment of the invention, the injection catheter has a distal stop, collar, or flange disposed in the catheter distal region for limiting penetration of the catheter distal tip. In some devices, the distal stop is disposed between about 1 and2 mm. from the catheter disial tip. The distal stop will act to assure consistent and accurate penetration depths of the catheter distal tip to a location between the two tissue layers to be separated.

In some methods of the present invention, the barrier fluid can be methyl cellulose, hyaluronic acid, hydroxypropylmethylcelullose, saline, or a combination of the compounds. A barrier fluid that is a liquid is preferably a material that is biocompatible and which will be remain between the tissue layers for time sufficient for the treatment energy to be applied. The hydropropylmethyl cellulose or hyaluronic acid may be diluted in water or saline and used at a concentration of between 0.1% to 5.0% for injection. In another method, carbon dioxide gas or other gas is used as the barrier fluid.

In one method of the present invention, a biocompatible, viscous, thermal insulator is injected at a depth to separate the mucosal layer from the submucosal layer or to separate the submucosal layer into two portions. When heat energy is applied to the mucosal layer, thermal damage to the submucosal layer or deeper portion of the submucosal layer, and muscularis layer, can be prevented or significantly reduced.

The barrier fluid utilized with certain embodiments of the present invention may be injected into the tissue of the organ to be treated alone, or with a connective tissue weakening agent, such as hyaluronidase. In combination with a connective tissue weakening agent, better separation can be achieved of one of the tissue layers from another. The connective tissue weakening agent may be mixed with the barrier fluid or injected into the tissue at the desired location immediately before injection of the barrier fluid.

In one method of the present invention, the barrier fluid injected is a non electrically-conducting or electrically-insulating viscous fluid. In electrical treatments, for example, in RF applications, a bipolar delivery device may be used. In the bipolar delivery device, current flows from one pole, through the tissue, and returns to the second pole. Where complete ablation of the mucosal layer or of the mucosal and superficial portion of the submucosa is desired, the current path preferably extends only through the mucosal layer or through the mucosal layer and part way through the submucosal layer, but no deeper. The presence of an electrical insulating layer can act to block current flow through the deeper layers.

In some methods of the present invention, an electrically-conducting fluid is used. In the example previously described, current flow is desired through the mucosal but not through all of the submucosal layer. The electrical-conducting fluid can act to conduct any current tending to extend deeper than the mucosal layer or superficial portion of the submucosa through the highly electrically-conductive fluid and then back into the mucosal layer and to the second pole of the bipolar ablation device. The path of electrical current would preferably go through the highly conductive fluid rather than deeper into the tissue and through a less conductive portion of the submucosal layer.

In another method embodiment of the present invention, utilizing optical energy, including but not limited to photodynamic therapy, an optically-reflecting or optically-absorbing fluid can attenuate or eliminate transmission of light energy through the barrier fluid and to the protected layer below. Where cryoblation therapy is used, a thermally insulating fluid can prevent the penetration of the ice ball formed by the cryoablation probe from penetrating too deeply. In still another method of the present invention, the barrier fluid may be a foam with a significant amount of dissolved air or other gas.

In yet another method embodiment of the present invention, the fluid has an acoustic impedance significantly different than tissue to create an acoustically reflecting interface, or is highly acoustically absorbing, to block or attenuate the transmission of ultrasonic energies. An example of a suitable ultrasonic blocking fluid includes micro bubbles. In still another method of the present invention, the fluid is primarily a gas, for example, carbon dioxide. The carbon dioxide can act as a thermal and electrical insulator, while aiding in separation of the layers.

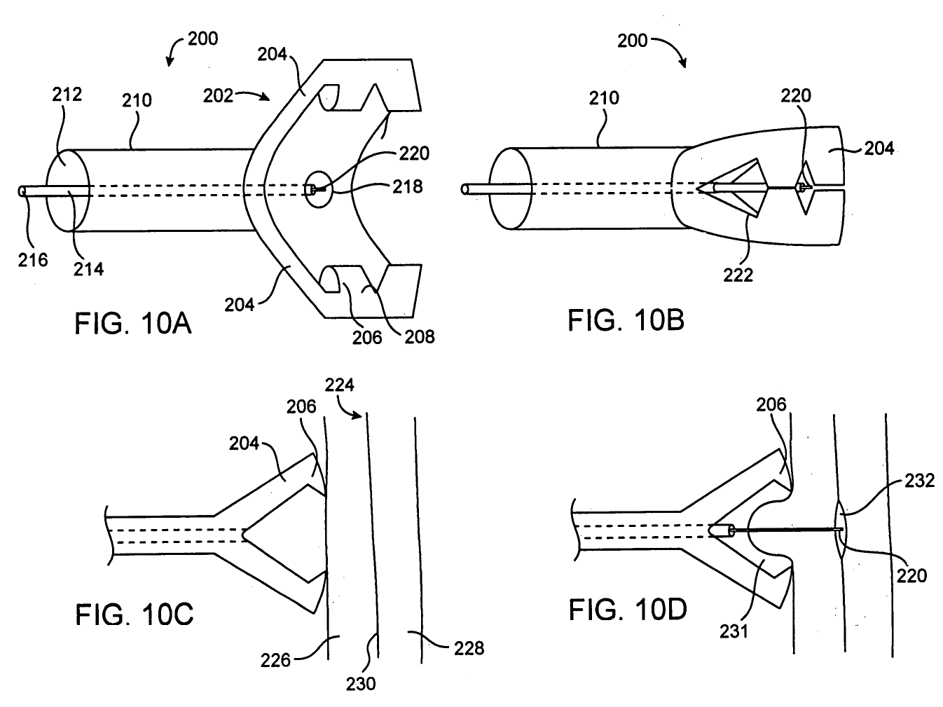

Referring now to FIG. 10(a), a distal region of a first barrier material injection device 200 is illustrated in an open position, and having closable opposed jaws 204 for grasping and retracting a tissue layer to create a potential space between the retracted layer and a deeper layer, and a longitudinally slidable needle or catheter 214 for injecting barrier material into the potential space. Barrier material injection device 200 includes a shaft 210 and a distal mechanism 202 for attaching to the lining of an organ and retracting the lining to allow separation of the tissue planes, Jaws 204 are mounted in an opposed fashion to each other and have distal teeth and a groove or opening 208 for allowing passage of an injection catheter or needle even while the jaws are in a closed position. Mechanism 202 includes a distal. port 218 for allowing distal extension of an injection needle or catheter there through.

An injection needle or catheter 214 can be slidably disposed within a lumen 212 formed within shaft 210, with catheter 214 having an injection lumen 216 therethrough. Injection needle 214 may be seen to have a distal. tip 220. Barrier material injection device 200 is shown in a first position in FIG. 10(a)., having jaws open and needle 214 retracted.

FIG. 10(b) illustrates barrier material injection device 200, having jaws 204 in a fully closed position, and showing jaw side openings 222. FIG. 10C is a side view of device 200, with jaws 204 in an open position, with injection needle 214 retracted, approaching a layered tissue 224 to be separated. Layered tissue 224 includes a superficial layer 226 and a deeper layer 228, separated by a tissue plane 230. Superficial tissue layer 226 has been approached by jaw teeth 206.

FIG. 10(d) further illustrates barrier material injection device 200, with jaws 204 closed about and retracting a tissue portion 231 to create a potential space 232, and having injection needle tip 220 inserted into potential space 232.

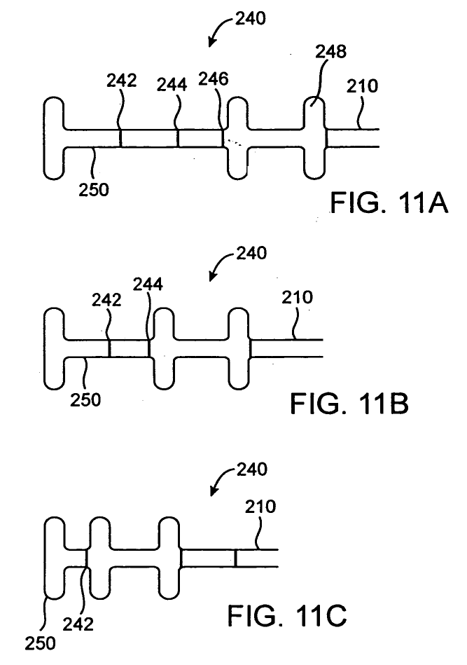

FIG. 11(a) illustrates one barrier injection device proximal handle portion 240 suitable for coupling to barrier material injection device 200. Proximal handle 240 includes markers or indents 246, 244, and 242, corresponding to the first, second, and third positions, respectively, as described with respect to FIGS. 10(a) through 10(d). Proximal handle 240 is shown in the first position, causing jaws 204 to open and injection needle 214 to retract. FIG. 11 (b) illustrates device proximal handle 240 shown in the second position, causing jaws 204 to close and injection needle 214 to retract.

FIG. 11 (c) illustrates device proximal handle 240, shown in the third position causing jaws 204 to close and needle 214 to distally extend. In some embodiments, the handle includes an inner shaft or tube 250 slidably disposed within an outer tube 210. In some embodiments, inner shaft 250 is coupled to outer tube 210 of FIG. 10A, and inner shaft 250 is coupled to injection catheter 214 of FIG. 10A.

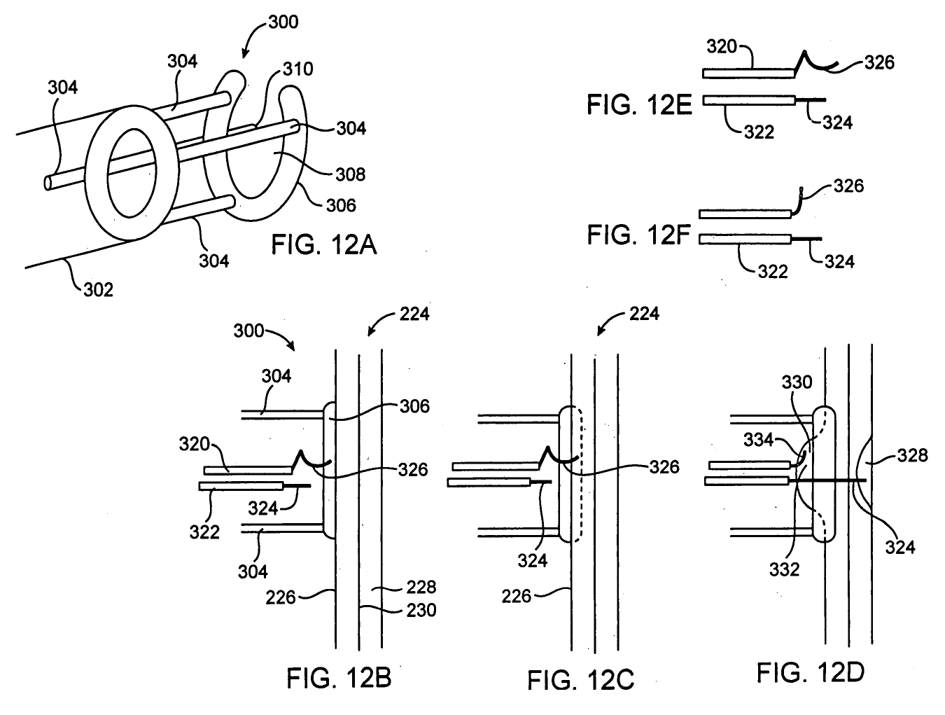

FIG. 12(a) illustrates another barrier material injection device 300 having a distal plate or ring 306 coupled to an outer tube 302, having an inner sheath 304 for slidably disposing a retraction catheter and/or needle, and an injection catheter and/or needle, within. Inner sheath 304 can terminate distally in a distal tip 310. Barrier material injection device 300 can include distal support members 304 for coupling distal plate 306 to outer tube 302.

FIG. 12(b) illustrates another barrier material injection device 300, having plate 306 disposed against superficial tissue layer 226. In this example, device 300 may be seen to include a retraction catheter or tube 320 having a retraction needle 326 extending distally therefrom. Barrier material injection device 300 also includes an injection catheter 322 terminating in a distal injection needle 324. In some devices, both injection catheter 322 and retraction catheter 320 are slidably disposed within inner sheath 304.

As illustrated in FIG. 12(c), barrier material injection device 300 is shown with retraction needle 326 penetrating and engaging superficial tissue layer 226. FIG. 12(d) shows device 300 with retraction needle 326 retracting superficial tissue layer 226 to create a potential space 328 along tissue p lane 230 between superficial tissue layer 226 and deeper tissue layer 228. Injection needle 324 has been inserted into potential space 328.

Referring to FIG. 12(e), retraction needle 326 and injection needle 324 are shown as one embodiment of a barrier material injection device. Retraction needle 326 is distally extended from within retraction catheter tube 320. In some embodiments, the retraction needle has a helical or pig tail distal region that can be rotated within a retraction tube to penetrate and engage the superficial tissue layer, and the retraction catheter and/or retraction needle proximally retracted to retract the superficial tissue layer. FIG. 12(f) shows retraction needle 326 retracted proximally into retraction tube 322. In some embodiments, the retraction catheter and needle can be formed as a shaft having a distal needle portion, with both being slidably as a single unit.

In one embodiment of the present invention, an endoscope can be inserted into the esophagus and positioned appropriately. A barrier material injection device, such as devices 200, 300, and the like is inserted through the working channel of the endoscope and positioned adjacent to the inner wall of the esophagus. The end of a barrier material injection device is manipulated to grab the superficial layers of the esophageal wall (mucosa and submucosa) and pull them away from the deeper layers (muscularis propria and connective tissue) in a tenting action. A needle is then inserted through the tented portion of the wall, between the superficial and deeper layers and the barrier material is inserted via injection. The barrier material can be placed between the second and third layer i.e. submucosa and muscle or between the first and second layer i.e. mucosa and submucosa. A potential space can be created between layers, which can then be expanded by insertion of a barrier material.

In another embodiment of the present invention, with use of a barrier material injection device such as devices 200 or 300, the tip has a small ring-like or U-shaped member with an attached and deployable needle to grab the superficial tissue layer, as discussed with respect to FIGS. 12(a) through 12(f). The needle can be recessed under the ring to allow passage of the device through a working endoscope channel. At the appropriate time, the needle can be pushed adjacent to or into the tissue to anchor the device, to allow tenting of the superficial layer away from the deeper layers via gentle endoscope manipulation and traction. A second injection needle can then be inserted into the center of the tented material, into the potential space created when the tissue layers are pulled apart. The barrier material can then injected into the tented space to create a true barrier.

In one method of the present invention, utilized barrier material injection device 200 illustrated in FIGS. 10(a) through 10(d), the tip of barrier material injection device 200 is made of opposing jaws 204, similar to some existing biopsy forceps, well known to those skilled in the art. In this embodiment, jaws 204 can be grooved in the center, and spaced appropriately to allow the injection needle to be extruded from a hollow catheter for injection of the barrier material. Jaws 204 grasp the superficial tissue in a pincer-like fashion, and the superficial tissue layers are then tented away from the deeper layers via gentle traction. A needle is then extruded from the hollow catheter, through the groove in the jaws and into the space created between layers. The barrier material/fluid is then injected.

In various embodiments of the present invention, the catheter of the barrier material injection device, such as devices 200 or 300, is hollow, allowing the injection needle to be recessed into the body of the catheter when not in use. The needle can be extruded when needed via a mechanism attached to the handle of the barrier device.

It will be appreciated that the present invention is not limited to the examples above as the only mechanisms to attach to superficial tissue layers to allow separation. Various methods of the present invention can utilize multiple variations of mechanisms, including the application of suction, to provide for the grasping or attachment to superficial tissue lining or layers and allow separation. In various embodiments of the present invention, the barrier material can be colored using methylene blue, food coloring or other substances, to identify the areas of the esophagus that have been adequately, or inadequately, injected.

The following non-limiting example illustrates certain embodiments of the present invention.

### EXAMPLE 2

Prior to ablating the esophagus 14 of a pig using the ablation device and catheter apparatus and devices described herein, an injection catheter was angled into the esophagus 14 to the location to be treated. The endoscope was angled so that the injection catheter could abut the tissue and a needle associated with the injection catheter could enter the tissue to a depth below the mucosa and above the muscle layer, about 1-2 mm into the tissue layers.

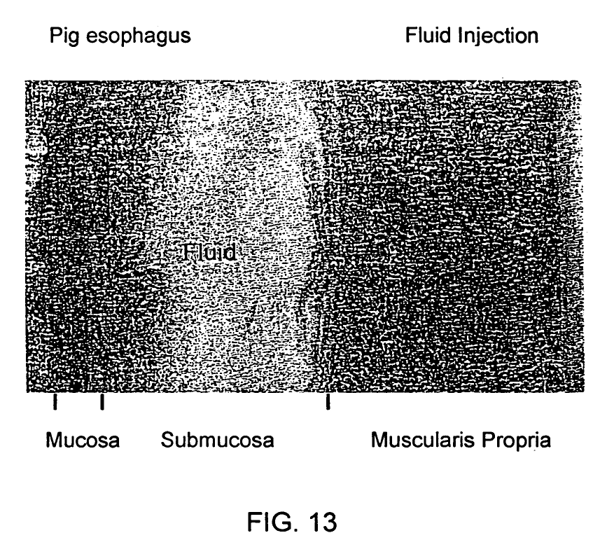

A barrier fluid, comprising about 0.5% hydroxypropylmethyl cellulose, was injected through the needle into the tissue. Barrier fluid was radially injected at four injection sites prior to ablation. FIG. 13 shows a photograph of a histologic specimen (pig) demonstrating the separation of esophageal layers (mucosa/submucosa from muscle) using a barrier fluid.. As can be seen from the photograph, in this example, the barrier fluid was injected into the submucosal layer, expanding the layer into a superficial portion adjacent the mucosa and a deeper portion adjacent the muscle layer.

In this particular experiment, 750 joules of energy with 90 watts of power were applied to the target area for up to ten seconds. The portion of the esophagus with the barrier material inserted below the mucosa and above a portion of the submucosa and deeper tissue revealed normal muscle upon visual inspection. Visual inspection of the portion of the esophagus without the barrier material revealed about 50% of the muscle was nonviable.

The foregoing description of a preferred embodiment of the invention has been presented for purposes of illustration and description. It is not intended to be exhaustive or to limit the invention to the precise forms disclosed. Obviously, many modifications and variations will be apparent to practitioners skilled in this art. It is intended that the scope of the invention be defined by the following claims and their equivalents.

What is claimed is:

1. A method of treating a tissue site of a tissue structure that has at least a first and a second tissue plane, comprising:
providing an energy delivery device;
creating a barrier between the first and second tissue planes;
positioning at least a portion of an energy delivery device at the tissue site; and
delivering sufficient energy from the energy delivery device to create cell necrosis of at least a portion of the first tissue plane.

2. The method of claim 1, wherein a barrier material is injected into a space between the first and second tissue planes.

3. The method of claim 2, wherein the barrier material attenuates transmission of energy delivered by the energy delivery device.

4. The method of claim 3, wherein the barrier material is a fluid.

5. The method of claim 4, wherein the fluid is a liquid fluid.

6. The method of claim 3, wherein the fluid is a gaseous fluid.

7. The method of claim 3, wherein the barrier material includes a connective tissue weakening agent for improving the separation of the tissue layers.

8. The method of claim 7, wherein the connective tissue weakening agent includes hyaluronidase.

9. The method of claim 4, wherein the fluid is thermally insulating.

10. The method of claim 4, wherein the fluid is electrically insulating.

11. The method of claim 4, wherein the fluid is electrically conducting.

12. The method of claim 4, wherein the fluid-tissue interface is ultrasonically reflecting or ultrasonically absorbing.

13. The method of claim 4, wherein the fluid is optically reflecting or absorbing.

14. The method of claim 1, further comprising:
providing an injection catheter for introducing the having a lumen therethrough, a distal region, an injection orifice disposed in the distal region and in fluid communication with the lumen.

15. The method of claim 14, further comprising:
injecting the barrier material in one or more radially spaced injection in a region of the tissue site.

16. The method of claim 15, wherein a plurality of radially spaced injections are made in the region of the tissue site, the plurality of radially spaced injections being spaced between about 0.5 and 2 centimeters.

17. The method of claim 14, wherein about 1 and 10 cubic centimeters of barrier material are injected at the tissue site.

18. The method of claim 14, wherein at least four radially spaced injections are made at the tissue site.

19. The method of claim 2, wherein the barrier material is colored with methylene blue or food coloring.

20. The method of claim 2, wherein the first tissue plane is the mucosa, the second tissue plane is muscle and wherein a third tissue plane is a submucosa that is located between the mucosa and muscle planes.

21. The method of claim 20, wherein the mucosa and submucosa tissue planes are separated from the muscle plane.

22. The method of claim 21, wherein the mucosa plane is separated from the submucosa plane.

23. The method of claim 2, wherein the tissue site is at a gastrointestinal or genitourinary organ.

24. The method of claim 2, wherein the tissue site is any tissue site with multiple tissue planes.

25. The method of claim 1, wherein the energy delivery device is coupled to an expandable member.

26. The method of claim 25, further comprising:
expanding the expandable member.

27. The method of claim 1, wherein the first tissue plane is a mucosal layer of the esophagus.

28. The method of claim 27, wherein the expandable member is expandable sufficiently to cause at least a portion of the energy delivery device to be in contact with the esophagus mucosal layer.

29. The method of claim 2, further comprising:
identifying an existence of abnormal tissue in the mucosal layer.

30. The method of claim 29, wherein the abnormal tissue is visually identified.

31. The method of claim 1, wherein the energy is delivered from an RF source.

32. The method of claim 1, wherein the energy is delivered from a microwave source.

33. The method of claim 1, wherein the energy is delivered from an optical source.

34. The method of claim 1, wherein the energy is delivered from an ultraviolet light source.

35. The method of claim 1, wherein the energy is delivered from a thermal source.

36. The method of claim 1, wherein the energy is delivered from a resistive heating source..

37. The method of claim 1, wherein the abnormal tissue is selected from Barrett's epithelium, variants of Barrett's epithelium, dysplastic tissue and malignant tissue.

38. A method of treating an esophagus tissue site with at least a first and a second tissue plane, comprising:
introducing an energy delivery device through an oral cavity and into the esophagus;
positioning at least a portion of an energy delivery device at the esophagus tissue site; and
creating a barrier between the first and second tissue planes;
positioning at least a portion of an energy delivery device at the esophagus tissue site; and
delivering energy from the energy delivery device at different times to create cell necrosis of at least a portion of the first tissue plane.

39. The method of claim 38, wherein a barrier material is injected into a space between the first and second tissue planes.

40. The method of claim 39, wherein the energy delivery device is coupled to an expandable member.

41. The method of claim 40, further comprising:
expanding the expandable member.

42. The method of claim 41, further comprising:
viewing the cell necrosis.

43. The method of claim 41, wherein the first plane of the tissue site is a mucosal tissue of the esophagus.

44. The method of claim 43, wherein the first plane of the tissue site is a mucosal tissue and a sub-mucosal tissue of the esophagus.

45. The method of claim 43, wherein the expandable member is expandable sufficiently to cause at least a portion of the energy delivery device to be in contact with the esophagus mucosal layer.

46. The method of claim 43, further comprising:
identifying an existence of abnormal tissue in the mucosal layer.

47. The method of claim 46, wherein the abnormal tissue is visually identified.

48. The method of claim 38, wherein the energy is delivered from an RF source.

49. The method of claim 38, wherein the energy is delivered from a microwave source.

50. The method of claim 38, wherein the energy is delivered from an optical source.

51. The method of claim 38, wherein the energy is delivered from an ultraviolet light source.

52. The method of claim 38, wherein the energy is delivered from a thermal source.

53. The method of claim 38, wherein the energy is delivered from a resistive heating source.

54. The method of claim 38, wherein the abnormal tissue is selected from Barrett's epithelium, variants of Barrett's epithelium, dysplastic tissue and malignant tissue.

55. A method of treating an esophagus tissue site with at least a first and a second tissue plane, comprising:
introducing an energy delivery device through an oral cavity and into the esophagus;
creating a barrier between the first and second tissue planes positioning at least a portion of an energy delivery device at the esophagus tissue site; and
delivering energy from the energy delivery device to create a controlled cell necrosis of at least a portion of the first tissue plane while minimizing permanent damage to esophageal muscularis tissue.

56. The method of claim 55, wherein a barrier material is injected into a space between the first and second tissue planes.

57. The method of claim 55, wherein the energy delivery device includes an expandable member.

58. The method of claim 57, further comprising:
expanding the expandable member.

59. The method of claim 55, further comprising:
viewing the cell necrosis.

60. The method of claim 55, wherein the tissue site is a mucosal tissue of the esophagus.

61. The method of claim 55, wherein the tissue site is a mucosal tissue and a sub-mucosal tissue of the esophagus.

62. The method of claim 57, wherein the expandable member is expandable sufficiently to cause at least a portion of the energy delivery device to be in contact with the esophagus mucosal layer.

63. The method of claim 60, further comprising:
identifying an existence of abnormal tissue in the mucosal layer.

64. The method of claim 56, wherein the abnormal tissue is visually identified.

65. The method of claim 55, wherein the energy is delivered from an RF source.

66. The method of claim 55, wherein the energy is delivered from a microwave source.

67. The method of claim 55, wherein the energy is delivered from an optical source.

68. The method of claim 55, wherein the energy is delivered from an ultraviolet light source.

69. The method of claim 55, wherein the energy is delivered from a thermal source.

70. The method of claim 55, wherein the energy is delivered from a resistive heating source.

71. The method of claim 55, wherein the abnormal tissue is selected from Barrett's epithelium, variants of Barrett's epithelium, dysplastic tissue and malignant tissue.

72. A method of treating an esophagus tissue site with at least a first and a second tissue plane, comprising:
introducing an energy delivery apparatus through an oral cavity and into the esophagus, the energy delivery apparatus including a plurality of RF electrodes, a width of each RF electrode and a spacing between adjacent RF electrodes selected to provide a selectable ablation of an esophagus mucosal tissue;
creating a barrier between the first and second tissue planes;
positioning at least a portion of an energy delivery device at the esophagus tissue site; and
delivering energy from the energy delivery device to create a controlled cell necrosis of at least a portion of the first tissue plane while minimizing permanent damage to a muscularis tissue.

73. The method of claim 72, wherein a barrier material is injected into a space between the first and second tissue planes.

74. The method of claim 72, wherein the plurality of RF electrodes are arranged in a pattern.

75. The method of claim 72, where at least a portion of the plurality of RF electrodes are bi-polar RF electrodes.

76. The method of claim 72, wherein a width of each RF electrode is no more than 3 mm.

77. The device of claim 72, wherein a width of each RF electrode is no more than 2 mm.

78. The device of claim 72, wherein a width of each RF electrode is no more than 1 mm.

79. The device of claim 72, wherein a width of each RF electrode is no more than 0.5 mm.

80. The device of claim 72, wherein a spacing between adjacent RF electrodes is no more than 2 mm.

81. The device of claim 72, wherein a spacing between adjacent RF electrodes is no more than 1 mm.

82. The device of claim 72, wherein a spacing between adjacent RF electrodes is no more than 0.5 mm.

83. The device of claim 72, wherein the plurality of electrodes are arranged in segments.

84. The device of claim 83, wherein at least a portion of the segments are multiplexed.

85. The device of claim 84, wherein an RF electrode between adjacent segments is shared by each of adjacent segments when multiplexed.

86. An apparatus for separating tissue planes at a tissue site, comprising,
an instrument body having an elongated shaft portion sized and constructed for insertion through an endoscope and into the interior of the tissue site of a patient;
a separation mechanism at a distal end of the elongated shaft that is attachable to at least a portion of the tissue site, the separation mechanism providing a separation of at least one tissue plane from a second tissue plane; and
an injection device coupled to the instrument body.

87. The apparatus of claim 86, wherein the separation mechanism includes two opposing jaws.

88. The apparatus of claim 87, wherein at least one of the two opposing jaws is grooved and spaced to permit passage of at least a portion of the injection device.

89. The apparatus of claim 86, wherein the separation mechanism is a ring or u-shaped member.

90. The apparatus of claim 89, wherein the ring or u-shaped member is adapted to allow passage of at least a portion of the injection device.

91. The apparatus of claim 86, wherein at least a portion of the instrument body is hollow and the injection device includes a recessed needle that can be extruded for injection.

92. A system for creating cell necrosis from a tissue site at a human esophagus, comprising:
a cell necrosis device;
an injection catheter for injecting a barrier material to separate esophageal tissue layers by flowing between tissue layers or expanding one of the tissue layers, the injection catheter having a lumen therethrough, a proximal region, a proximal port, a distal region, and an injection orifice at a distal region and in fluid communication with the lumen; and
a fluid supply coupled to the injection catheter proximal port for forcing the barrier fluid through the injection catheter distal orifice.

93. The system of claim 92, wherein the injection catheter has a stop disposed in the catheter distal region for limiting penetration of the catheter into the esophageal tissue.

94. The system of claim 93, further comprising an endoscope adapted to admit the injection catheter therethrough.

95. The system of claim, 92 wherein the energy therapy further includes the use of a drug sensitizer.

96. The method of claim 1, further comprising:
viewing the cell necrosis.
